(19) United States
(12) Patent Application Publication
(10) Pub. No.: US 2002/0127716 A1
(43) Pub. Date: Sep. 12, 2002

(54) Methods of preparing olfactory ensheathing cells for transplantation

(76) Inventors: Francois Feron, (US); Alan MacKay-Sim, (US)

Correspondence Address: GREGORY P. EINHORN, Fish & Richardson P.C., 4350 La Jolla Village Drive, Suite 500, San Diego, CA, 92122, US

(21) Appl. No.: 10/134,141

(22) Filed: Apr. 26, 2002

Related U.S. Application Data
(63) Continuation of application No. PCT/AU00/01327, filed on Oct. 27, 1999.

(30) Foreign Application Priority Data
Oct. 27, 1999 (AU) AU1999PQ03695

(51) Int. Cl. C12N 5/08; A61K 45/00

(52) U.S. Cl. 435/368; 424/93.7

(57) ABSTRACT

A method is described for isolating ensheathing cells, in particular those from olfactory lamina propria and use of the isolated ensheathing cells and lamina propria respectively in transplantation. Isolated lamina propria and ensheathing cells from the olfactory mucosa are well suited for autologous transplantation, where the donor and recipient are the same, as surgical biopsy of the olfactory mucosa is less damaging than isolating tissue from other location of a person's body, for example the olfactory bulb. Transplantation is particularly directed to neural regions (for example the brain, spinal cord and/or peripheral nerves) of a human to assist recovery of acute and chronic nerve damage following surgery or trauma.

### RELATED APPLICATIONS

This application is a continuation of international patent application serial No. PCT/AU00/01327, filed Oct. 27, 2000, which has as a priority document Australian patent application serial no. AU1999PQ03695, filed Oct. 27, 1999. Each of the aforementioned applications is explicitly incorporated herein by reference in their entirety and for all purposes.

### FIELD OF THE INVENTION

This invention relates to a method of isolating ensheathing cells, e.g., from isolated olfactory lamina propria, and use of the isolated ensheathing cells or isolated lamina propria in transplantation. The invention has particular application in autologous transplantations directed to neural regions (for example brain, spine and/or peripheral nerves) of a human to assist recovery of acute and chronic nerve damage following surgery or trauma.

### BACKGROUND OF THE INVENTION

Olfactory mucosa comprises at least two anatomically distinct cell layers: olfactory epithelium (comprising of supporting cells, basal cells, immature neurons and mature sensory neurons) and lamina propria (comprising of ensheathing, glial cells, endothelial cells, fibroblasts or glandular cells). Olfactory ensheathing cells enwrap axons of olfactory nerves in olfactory nerve bundles in the lamina propria and in the olfactory bulb; the olfactory bulb is the site of olfactory nerve axon termination in the brain. The olfactory ensheathing cells are specialized glia which have two interesting and useful properties. Like Schwann cells of the peripheral nervous system, ensheathing cells permit and promote axon growth, properties not seen in the glia of the central nervous system. However, unlike Schwann cells, olfactory ensheathing cells exist both within and outside the central nervous system.

In the last few years several studies have been published which indicate that functional repair of the spinal cord might be possible and that peripheral nerve repair might be improved. A key to the reported successes is the transplantation of ensheathing cells from the olfactory nerve layer of the olfactory bulb (reviewed: Doucette, 1995, Histol Histopathol 10 503; Fawcett, 1998, Spinal Cord 36 811; Lu and Waite, 1999, Spine 24 926; Ramon-Cueto and Avila, 1998, Brain Res Bull 46 175).

Transplants of olfactory nerve ensheathing cells from the olfactory bulb promote regeneration of parts of the central nervous system which do not normally regenerate: entry of dorsal root axons into the spinal cord (Ramon-Cueto and NietoSampedro, 1994, Exp Neurol 127 232), regeneration of corticospinal axons after electrolytic lesion (Li et al. 1998, Jour Neurosci. 18 10514), remyelination of the dorsal columns after x-ray irradiation (Imaizumi et al, 1998, Jour Neurosci 18 6176) and regeneration of spinal cord axons through Schwann cell-filled guidance channels (Ramon-Cueto et al, 1998, Jour Neurosci 18 3808). Olfactory ensheathing cell transplants from the olfactory bulb have allowed some functional recovery after corticospinal tract lesion (Li et al, 1997, Science 277 2000). However, other publications describe olfactory bulb ensheathing cells assisting peripheral nerve regrowth, but fail to demonstrate functional recovery (Verdu et al, 1999, Glia 10 1097). This may have been due to the source and state of the cells. These cells were dissociated from the olfactory bulb, immunopurified, (sometimes stored frozen and thawed) and then used for grafting. The method disclosed in this publication is unsatisfactory and may damage the cells, killing many of them and stressing the remainder.

Published studies of ensheathing cell transplants have removed cells from an exterior layer of the olfactory bulb in the brain of a donor and transplanted the cells into a different recipient. For human therapy it has been suggested that ensheathing cells could be harvested post-mortem or from embryos (Navarro et al, 1999, Ann Neurol 45 207); however, use of embryonic tissue is ethically questionable and use of post-mortem tissue may be complicated by cell or tissue rejection. Further, use of cells isolated from the olfactory bulb for autologous transplantation in humans is of limited value because of the difficulty and likely damage to the brain when collecting a biopsy sample.

An alternative source of olfactory neural tissue other than the olfactory bulb is the olfactory mucosa. Methods of isolating and culturing rat olfactory epithelium and lamina propria is disclosed in Feron et al, 1999, Neuroscience 88 571, herein incorporated by reference. This document discloses methods of purifying basal cell cultures from adult rat olfactory epithelium, culturing the cells in either serum-free (for epithelium containing basal and supporting cells) or serum-containing (for lamina propria) medium and inducing the basal cells to differentiate into neurons using biochemical or mechanical stress.

International publication WO98/12303 describes a method of culturing a mixed population of cells from a tissue sample that includes a heterogeneous population of neuronal and glial cells from neonatal rat olfactory neuroepithelial tissue. This mixed population of cells is used for screening neuronal growth factors, neuroprotective agents, neurotoxins, therapeutic or prophylactic agents and agents that affect cell activity. This document does not disclose methods for isolating and culturing ensheathing cells.

### OBJECT OF THE INVENTION

The present inventors have realized limitations of mixed cell cultures of neurons and ensheathing cells, particularly for use in procedures such as transplantation where only a subset of cell types may be desired. The present invention relates to a method of preparing isolated ensheathing cells, particularly from olfactory lamina propria, for transplantation. The separation and removal of the olfactory epithelium (containing nerve and basal cells) from the lamina propria (containing ensheathing cells) has advantages when compared to culturing a mixed population of neurons and ensheathing cells. The prior separation and isolation of the lamina propria provides a means for enriching for ensheathing cells and the enriched cell population may then be more efficiently purified using methods including the step of immunopurification. It is also important to remove epithelial basal cells that once transplanted into a nerve might induce a cyst or tumour.

It is therefore an object of the invention to provide a method of isolating ensheathing cells from olfactory lamina propria and preparing and using the lamina propria or ensheathing cells therefrom for transplantation.

### SUMMARY OF THE INVENTION

An aspect of the invention relates to a method of isolating ensheathing cells comprising the steps of:

(i) isolating olfactory mucosa;

(ii) isolating lamina propria from the isolated olfactory mucosa; and

(iii) isolating ensheathing cells from the isolated lamina propria.

In one aspect, the isolated olfactory mucosa of step (i) is isolated from the dorso-medial area of a nasal septum or superior turbinate or middle turbinate proximal to the cribriform plate.

In one aspect, the olfactory mucosa is isolated from an adult.

The olfactory mucosa may be isolated from a mammal.

In one aspect, the mammal is a human.

In one aspect the isolation of ensheathing cells includes the steps of:

(a) isolating olfactory mucosa;

(b) enzymatic digestion of the isolated olfactory mucosa; and

(c) mechanical separation of the lamina propria from the olfactory epithelium.

In one aspect, the enzymatic digestion of step (b) includes digestion with dispase II.

Another aspect of the invention relates to a method of isolating ensheathing cells including the steps of:

(I) isolating lamina propria from olfactory mucosa;

(II) enzymatically digesting the isolated lamina propria of step (I); and

(III) isolating ensheathing cells from the enzymatically digested isolated lamina propria of step (II).

In one aspect, step (II) includes collagenase L and dispase II.

In one aspect, step (II) includes the enzyme collagenase L.

In yet another aspect, the invention relates to a method of isolating ensheathing cells including the steps of:

(A) isolating lamina propria from olfactory mucosa;

(B) slicing and culturing the isolated lamina propria;

(C) allowing ensheathing cells to migrate away from the cultured lamina propria; and

(D) isolating the ensheathing cells.

A suitable thickness of the isolated lamina propria of step (B) is about 200 to 400 μm.

In still yet another aspect of the invention relates to a method of isolating ensheathing cells including the step of isolating ensheathing cells bound by an antibody that binds ensheathing cells.

In one aspect, the method includes the step of immuno-panning, immunoprecipitation or a combination thereof.

In one aspect, immunoprecipitation includes the step of using magnetic beads whose surface is coated with a secondary antibody that binds to the antibody that binds the ensheathing cells.

The antibody that binds ensheathing cells can be a monoclonal antibody that binds p75.

A further step may be included for culturing the antibody bound ensheathing cells in a culture medium supplemented with at least one of the following: epidermal growth factor, basic fibroblast growth factor, brain-derived neurotrophic factor, neurotrophic growth factor, neurotrophin 3, platelet-derived growth factor A, platelet-derived growth factor B, transforming growth factor α, leukemia inhibitory factor, ciliary neurotrophic factor or insulin-like growth factor-I.

Ensheathing cells may be expanded by culturing with conditioned medium from an olfactory lamina propria cell culture.

In one aspect, the olfactory lamina propria cell culture comprises cells other than ensheathing cells.

Yet still further, the invention relates to a method of transplanting ensheathing cells including the steps of:

(A″) isolating olfactory ensheathing cells; and

(B″) transplanting the isolated ensheathing cells of step (A″) to a recipient.

The ensheathing cells of step (A″) can be isolated from lamina propria of olfactory mucosa.

In still yet a further aspect, the invention relates to a method of isolating lamina propria including the steps of:

(A′) isolating olfactory mucosa from a human; and

(B′) isolating lamina propria from the isolated olfactory mucosa.

In still yet a further aspect, the invention relates to a method of transplanting lamina propria including the steps of:

(I′) isolating olfactory lamina propria from olfactory mucosa of a donor; and

(II′) transplanting the isolated olfactory lamina propria of step (I′) to a recipient.

The lamina propria may be intact or dissociated.

Transplantation may be heterologous or autologous.

In one aspect, the transplantation is autologous.

In one aspect, the donor or recipient is an animal.

In one aspect, the animal is a mammal.

In one aspect, the mammal is a human.

Transplantation may be to any organ or tissue of the recipient capable of neural growth.

In one aspect, the organ or tissue has nerve damage.

In one aspect, the organ or tissue with nerve damage is selected from the group consisting of brain, spine and peripheral nerves.

Throughout this specification unless the context requires otherwise, the word “comprise”, and variations such as “comprises” or “comprising”, will be understood to imply the inclusion of the stated integers or group of integers or steps but not the exclusion of any other integer or group of integers.

### BRIEF DESCRIPTION OF THE FIGURES

FIG. 1 is a photographic representation showing human nasal distribution of ensheathing cells in dorso-medial areas of the nasal cavity close to the cribriform plate. The large image is a scan of the nasal cavity and the insets show human ensheathing cells visualised using an anti-primate p75 antibody in tissue sections taken from biopsies removed from the regions indicated by the arrows.

FIGS. 2A and 2B are photographic representations showing cultures of human ensheathing cells visualised using an anti-primate p75 antibody. FIG. 2A shows a culture of dissociated cells. The culture is a mixture of p75-positive ensheathing cells (dark cells) and unstained cells seen here using Hoffman optics to increase their visual contrast. FIG. 2B shows p75-positive ensheathing cells migrating away from a lamina propria explant (at the bottom of the photograph).

FIG. 3 is a graph showing the numbers of ensheathing cells when cultured in DMEM comprising selected growth factors and on a substrate of plastic.

FIG. 4 is a graph showing the purity of ensheathing cell cultures when grown in DMEM comprising selected growth and on a substrate of plastic.

FIG. 5 is a graph showing the numbers of ensheathing cells when cultured in Neurobasal Medium comprising selected growth factors and on a substrate of fibronectin.

FIG. 6 is a graph showing the purity of ensheathing cell cultures when grown in Neurobasal Medium comprising selected growth factors and a substrate of fibronectin.

FIG. 7 is a photographic representation showing nerve regrowth after ensheathing cell grafting. The photographs show two nerves that have been sectioned. A nerve gap of 17 mm is replaced by a silicon tube. The upper photograph shows a nerve and tube into which ensheathing cells were transplanted and the nerve allowed to recover. The arrow indicates the regrowing nerve within the silicon tube. The lower photograph shows a control nerve and tube without ensheathing cell transplantation for which there is no nerve regrowth.

FIG. 8 shows recovery of hind limb movement after complete spinal cord transection and transplantation with olfactory lamina propria. FIGS. 8A-D are sequential frames of video images of an animal 8 weeks after transplantation showing flexion of the left ankle, knee and hip joints as the limb is moved forwards during walking on a 45° incline ladder. FIG. 8E is a histogram showing the mean BBB score (mean ±SE) for the best leg for respiratory lamina propria-transplanted animals (RLP), collagen matrix control animals (Con), olfactory lamina propria-transplanted animals (OLP), and dissociated olfactory ensheathing cell transplanted animals (OEC) 10 weeks (OLP) and 8 weeks (OEC, RLP, Con) after transplantation. FIG. 8F is a time course of functional recovery as assessed by the BBB score (mean ±SE) for control, OEC and OLP-transplanted animals and for 3 OLP-transplanted animals whose spinal cords were retransected 10 weeks after transplantation.

FIG. 9 shows functional recovery of descending suppression of spinal reflexes. FIG. 9A shows traces of EMG waves recorded from the 4th dorsal interosseous muscle in response to stimulation of the lateral plantar nerve. Upper pair of tracings (1), normal rat; middle pair of tracings (2) from a transected rat transplanted with respiratory lamina propria 10 weeks previously; lower pair tracings (3) from a transected rat with an olfactory lamina propria (OLP) transplant 10 weeks previously. The traces on the right are the responses to the first stimulus (control pulse) and on the left to the second of a train of stimuli at 10 Hz (test pulse after 100 ms interval). The black arrows indicate the position of the stimulus artifact and in each trace the M-wave (EMG response to stimulation of motor axons) is followed by an H-reflex (reflex response to stimulation of sensory axons). The H-reflex amplitude to the 2nd stimulus is depressed in normal and OLP-transplanted animals (white arrows). FIG. 9B is a histogram showing the H-reflex amplitude of the 2nd response (mean and SD, expressed as a percentage of the 1st response amplitude) for normal animals, animals transected with respiratory lamina propria and animals transected with OLP transplants. Each group is significantly different from the other 2 groups (normal versus both transected groups, p<0.01; transected control versus OLP-transplant animals, p<0.05).

FIG. 10a-10c shows regeneration of axons was promoted by olfactory lamina propria grafts. FIG. 10a shows a horizontal section through the graft site in an olfactory lamina propria-transplanted animal. The graft (G) integrated well with the rostral (R) and distal (D) cord. The region of the grafted tissue is shown by the bracket. FIG. 10b shows a high-power view within the olfactory lamina propria graft showing neurofilament immunoreactivity. At this focal plane many neurofilament-positive axons can be observed (arrows). FIG. 10c shows cell bodies in the nucleus raphe magnus were labeled retrogradely after injection of Fluororuby in the spinal cord caudal to the olfactory lamina propria graft. V marks the ventral edge of the medulla and the small arrows indicate labeled cell bodies. No cells were labeled after injections of Fluororuby caudal to respiratory lamina propria grafts. Scale bars: a, 1 □m; b, 100 □m; c, 10 □m.

FIG. 11 shows serotonergic fibres were present caudal to the olfactory lamina propria graft. FIGS. 11a and 11c show horizontal sections through the spinal cord rostral to the transplantation site. FIG. 11a is after respiratory lamina propria transplantation and FIG. 11c is after olfactory lamina propria transplantation. Serotoninergic positive axons are evident throughout the grey matter (Gr, arrows) and within the white matter (W, arrowheads). FIGS. 11b and 11d show horizontal sections through the spinal cord caudal to the transplantation site. FIG. 11b is after respiratory lamina propria transplantation and FIG. 11d is after olfactory lamina propria transplantation. Serotoninergic positive axons are evident only after olfactory lamina propria transplantation (FIG. 11d) at the border between the grey matter (arrows) and within the white matter (arrowheads). Scale bar: 50 μm.

### DETAILED DESCRIPTION OF THE EMBODIMENTS

In practice, ensheathing cells are usually isolated from the olfactory bulb of the brain. The present inventors have realised that there is an important and essential distinction between isolating the lamina propria and ensheathing cells originating from the olfactory mucosa and the usual site of isolating ensheathing cells from the olfactory bulb. In particular, for application in human transplantation, biopsy of the olfactory mucosa is a relatively painless procedure which does not affect the sense of smell and is acceptable to patients and research subjects (Féron et al, 1998, Archives of Otolaryngology Head and Neck Surgery 124 861, herein incorporated by reference). Ensheathing cells from the mucosa are therefore proposed as being ideally suited for autologous transplants in patients with brain injury, spinal injury, sensory and motor nerve injuries or after necessary nervous system damage during surgery.

This invention relates to a method of isolating ensheathing cells, in particular from olfactory lamina propria, and preparing and using the isolated ensheathing cells and lamina propria for transplantation to repair brain, spine and sensory and motor nerves following major trauma or surgery, for example to the head and neck. The methods comprise of grafting olfactory lamina propria, and ensheathing cells therefrom, into a region of nerve damage. These grafted ensheathing cells are “glia” or “helper” cells of the olfactory nerve. These olfactory ensheathing cells are chosen because they normally assist in the continual regeneration of olfactory nerves which occurs throughout life. This characteristic of the ensheathing cell may be useful in assisting nerve repair in a traumatised region. Further, because olfactory ensheathing cells are relatively accessible, these cells could be directly transplanted, or first isolated, from the nose of a patient at the time of definitive nerve repair. The invention has application to adult tissue which is a likely source of ensheathing cells in autologous transplantation involving a human patient. Isolation and culturing of adult tissue may be more difficult than culturing cells and tissue from neonates and the invention provides methods relating to adult tissue.

Ensheathing cells from the olfactory mucosa are very effective in promoting regrowth of axons across the resected spinal cord, with an attendant partial recovery of function after paralysis in rat. In monkey, autologous transplantation of olfactory lamina propria into hemisectioned spinal cord showed recovery from paralysis. These studies indicate that autologous lamina propria transplants and possibly ensheathing cells may be useful for repair of peripheral sensory and motor nerves and are discussed in more detail hereinafter.

Cells of the olfactory lamina propria, particularly ensheathing cells, have the advantage of being easily accessible from a nasal biopsy, obviating histocompatibility and rejection problems as well as avoiding many of the ethical issues in organ transplantation, particularly those involving embryonic stem cells and the adult human brain. Autologous transplantation also obviates technical and clinical problems associated with foreign tissue grafts.

In the case of lamina propria transplantation there is no requirement to isolate or purify the ensheathing cells. Grafting success might be dramatically improved if the cells do not undergo stressful procedures of purification as described in Verdu et al, 1999, supra. This can be avoided by using transplants of intact olfactory lamina propria. Another advantage of lamina propria grafts is that the tissue itself provides a substrate to support the grafted cells as well as providing a substrate through which the regenerating axons can grow. The olfactory lamina propria is a ready-made connective tissue matrix, largely collagen but consisting of other extracellular matrix molecules. A previous study has already demonstrated that a collagen matrix is more effective in supporting axon regrowth than a laminin gel (Verdu et al, 1999, supra). The intact lamina propria thus supplies two requirements for axon regrowth, ensheathing cells and a supportive matrix.

For human therapy, large numbers of olfactory ensheathing cells may be necessary for transplantation, so to limit the size of the biopsy and thus preserve the sense of smell of the patient it may be necessary to limit the amount of olfactory mucosa removed. This may require the in vitro proliferation of ensheathing cells prior to transplantation to expand the number of cells available for transplantation. Methods disclosed herein refer to the isolation of ensheathing cells from olfactory lamina propria and transplantation of the isolated ensheathing cells or lamina propria.

So that the invention may be understood in more detail the skilled person is directed to the following non-limiting examples.

### EXPERIMENTAL

1. Lamina Propria Isolation

Lamina propria isolation from rat was performed essentially as described in Feron et al, 1999, supra which is herein incorporated by reference. Briefly, a posterior part of a nasal septum of an anaesthetised adult rat was dissected free of the nasal cavity and immediately placed in ice-cold Dulbecco's modified Eagle's medium (DMEM) containing 50 mg/ml gentamicin and 10% (v/v) fetal calf serum. Cartilage of the septum was removed and the olfactory mucosa was incubated for 30 minutes at 37° C. in a 2.4 units/ml dispase II solution as previously described for skin (Roberts and Burnt, 1985, Biochem 232 67) and olfactory epithelium (Feron et al, 1995, J Neurosci Meth 57:9), herein incorporated by reference. The olfactory epithelium was carefully separated from the underlying lamina propria under a dissection microscope and the lamina propria was cultured in serum-containing medium to produce cultures of ensheathing cells.

Lamia propria cultures were centrifuged and the cell pellet was resuspended in DMEM comprising 10% fetal calf serum and gentamicin (50 mg/ml). Cells were seeded on glass cover slips and maintained at 37° C. and 5% CO₂.

It is appreciated that ensheathing cells may be isolated from olfactory mucosa without first isolating the lamina propria; however, the step of isolating the lamina propria may be preferred as this step enriches for ensheathing cells.

2. Collection of Biopsy Samples

The intranasal distribution of the human olfactory epithelium has previously been mapped (Feron et al, 1998, Arch. Otolaryngol Head Neck Surg 124 861). The probability of locating olfactory epithelium in a biopsy specimen ranges from 30% to 76%; the dorsoposterior regions of the nasal septum and the superior turbinate provide the highest probability of locating olfactory epithelium. These findings were partially confirmed in Leopold et al, 2000, Laryngoscope 110 417. However, a need to collect ensheathing cells in every single nasal biopsy led the inventors to perform another mapping to identify regions with a higher probability of successfully locating ensheathing cells. Since olfactory axons have to cross the cribiform plate of the ethmoid bone before synapsing in the olfactory bulb, the inventors hypothesized that the nerve surrounding cells, namely ensheathing cells, were present in high number in the area adjacent to this delineation.

Fifteen biopsies specimens were obtained from five human adult patients, aged 25 to 72 years. Nasal mucosa was obtained by biopsy during routine nasal surgery under general anesthesia, using an ethmoid forceps. The patients were undergoing surgery for septoplasty or turbinectomy. All samples were obtained under a protocol which was approved by the ethics committees of the hospital and university involved. All biopsy tissues were obtained with the informed consent of the patients and the studies were carried out in accordance with the guidelines of the National Health and Medical Research Council of Australia. Three areas of collection were chosen: the dorso-medial area of the superior turbinate, the dorso-medial area of the middle turbinate and the dorso-medial area of the septum. Biopsies were immediately fixed in a solution of 4% paraformaldehyde for 2 hours, washed in phosphate-buffered saline (pH 7.4), incubated in a 30% sucrose solution for 48 hours, frozen, sectioned at 8 μm and laid on slides coated with 3-aminopropyltriethoxy-silane (APES).

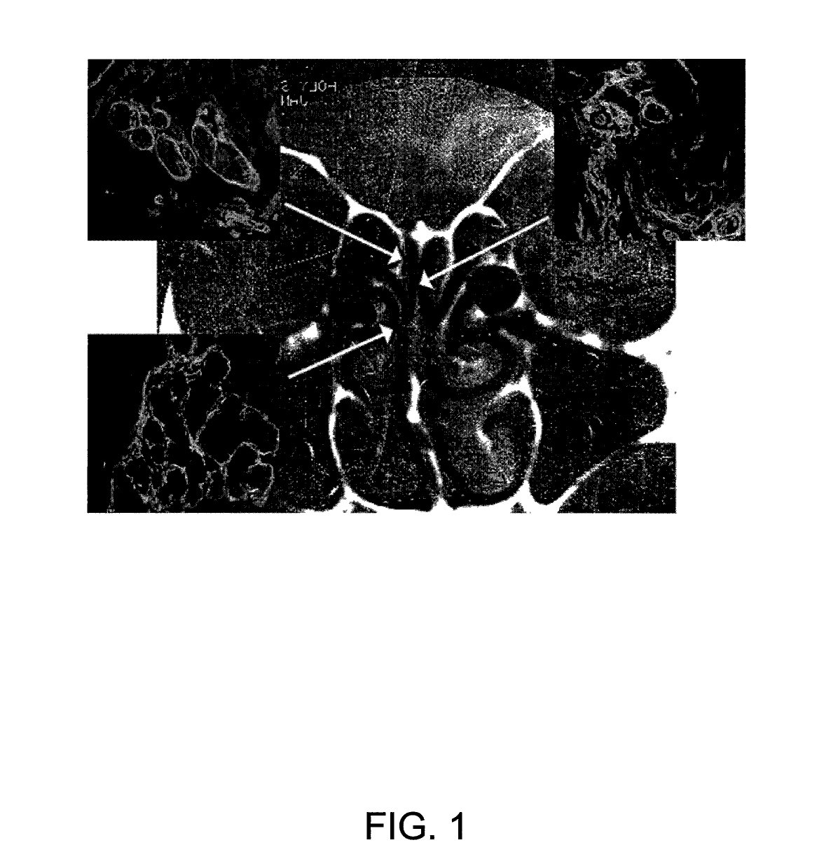

To detect the presence of ensheathing cells, immunochemistry was performed using two specific glial markers: anti-glial fibrillary acidic protein (GFAP) and anti-primate low affinity nerve growth factor receptor (p75) antibodies. Fluorescent or peroxidase conjugated secondary antibodies were used. FIG. 1 shows that ensheathing cells are found in all three areas inspected. However, higher density of ensheathing cells was found on the dorso-medial area of the septum. The central image of FIG. 1 represents a scanned cross section of a human nasal cavity. Biopsies were collected on the septum (right image), on the superior turbinate (top left image) or on the middle turbinate (bottom left image). Each peripheral image represents a section of the olfactory mucosa stained with a fluorescent p75 antibody.

3. Isolation and Culture of Ensheathing Cells

As previously described (Feron et al, 1999, supra), mammal olfactory epithelium and lamina propria were separated using the enzyme dispase II. Biopsies were placed in ice-cold Dulbecco modified Eagle's medium (DMEM) containing 50 mg/ml gentamicin and 10% (v/v) fetal calf serum and then incubated for 30 min at 37° C. in a 2.4 units/ml dispase II solution. The olfactory epithelium was carefully separated from the underlying lamina propria under the dissection microscope. Lamina propria tissues collected after dispase II incubation were enzymatically dissociated using a 0.025% solution of collagenase I for 15 minutes at 37° C. Enzyme activity was stopped with a Ca- and Mg-free buffer or with DMEM containing 0.53 mM ethylene-diamine-tetra-acetic acid (EDTA) solution and the suspension was centrifuged. The cell pellet was resuspended in the medium described above and cells were seeded on plastic Petri dishes.

Because the human olfactory mucosa is thicker and more compact compared with rat olfactory mucosa, especially in older patients, collagenase I was not able to fully dissociate the lamina propria. Five different combinations of enzymes were tested with various concentrations of components; a mixture of collagenase L (Sigma; 1 mg/ml) and dispase II (2.4 units/ml) was found to be most efficient. This combination is therefore recommended for the culture of dissociated human ensheathing cells. Collagenase I may be substituted for collagenase L for use with rat tissue.

Although more efficient than all the other combinations tested, this combination was not always able to achieve a complete dissociation of human lamina propria. To overcome this difficulty, an alternative technique was used: after removal of the olfactory epithelium, lamina propria pieces were sliced (200 μm thickness) using a McIlwain chopper (Brinkmann, N.Y., USA) before being transferred to fibronectin- or poly-L-lysine-coated plastic Petri dishes and cultured in the conditions above (Feron et al, 1998, supra). It was found that fibroblasts and endothelial cells grew quickly out of the explant during the first week, forming a bed cell layer. One week after initial plating, ensheathing cells migrated out of the explant crawling on the underlying cell layer of fibroblasts and endothelial cells. In the case of autologous transplantation, blood serum may be collected from a patient and used to culture the lamina propria slices.

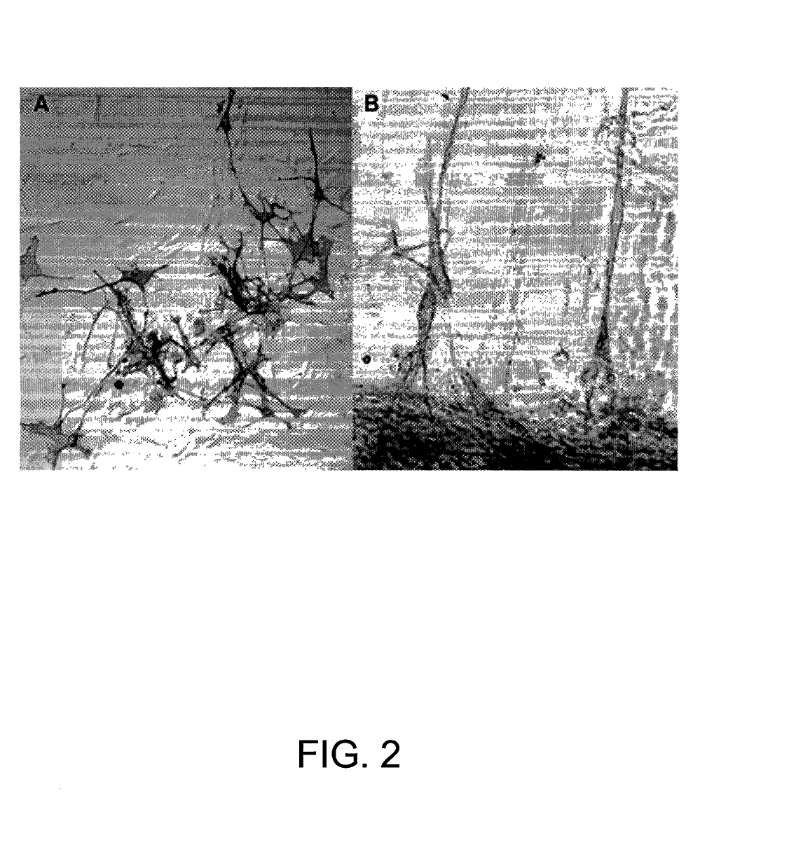

FIG. 2 shows cultures of human ensheathing cells. After removal of the olfactory epithelium, the lamina propria was either dissociated with a combination of collagenase and dispase (a, left) or sliced (b, right) and cultured in a serum containing medium for 10 days. Ensheathing cells were visualised using the anti-primate p75 antibody.

4. Purification of Ensheathing Cells

After cultivation for three weeks in a serum-containing medium, ensheathing cells were harvested using a combination of trypsin and EDTA, centrifuged at 300 g for 5 minutes and purified using three different techniques.

1. Immuno-panning. This method is based on a method described in Ramon-Cueto et al, 1998, J. Neuroscience 18 3803 wherein ensheathing cells were isolated from the olfactory bulb. The method includes the steps of incubating Petri dishes with 1:1000 biotinylated anti-mouse IgG antibody for 12 hours at 4° C. and washing the dishes three times with PBS. The dishes are then incubated with supernatants of cultured 192 hybridoma cells containing p75 low affinity nerve growth factor receptor (NGFR) antibody at 1:1 dilution in PBS with 5% bovine serum albumin for 12 hours at 4° C. After several washes with PBS, the cell suspension is plated on the antibody-treated dishes for 45 minutes at 37° C. Unbound cells are removed and the dishes are washed with a serum-free medium. Bound p75 expressing ensheathing cells are collected with a cell scraper, replated onto another antibody-treated dish and cultivated with DMEM containing a combination of EGF (25 ng/ml) and FGF (5 ng/ml).

2. Magnetic beads. The method is based on a method described by Barnett (Barnett et al, 2000, Brain 123 1581) and includes the steps of incubating attached cells from the above immuno-panning method with supernatants of cultured 192 hybridoma cells containing p75 NGFR antibody for 15 minutes at 37° C. before collection. After collection, the cell suspension is incubated with a solution of anti-mouse coated beads (Dynal), rotated for 5 minutes at 4° C. and bead-bound cells are separated using a magnet. After three washes in DMEM, purified ensheathing cells are resuspended, plated on a plastic culture dish and fed with DMEM containing a combination of EGF (25 ng/ml) and FGF (5 ng/ml).

3. Serum-free medium. To limit cell loss inherent to the previous methods (1 and 2 above) a new method of purification based on serum-free media was used. Following cell collection, the method includes the steps of, centrifuging and resuspending the cell suspension in either DMEM or Neuralbasal Medium (Gibco)—supplemented with one of the following growth factors: epidermal growth factor (EGF), basic fibroblast growth factor (FGF2), brain-derived neurotrophic factor (BDNF), nerve growth factor (NGF), neurotrophin 3 (NT3), platelet-derived growth factor A (PDGFA), platelet-derived growth factor B (PDGFB), transforming growth factor a (TGFa), insulin-like growth factor-I (IGF), leukemia inhibitory factor (LIF), or ciliary neurotrophic factor (CNTF). Cells were grown on either plastic culture dishes or plastic culture dishes coated with fibronectin (50 μg/ml). After seven days in culture, the cells are stained with an anti-glial fibrillary acidic protein (GFAP) or an anti-p75 antibody and counted.

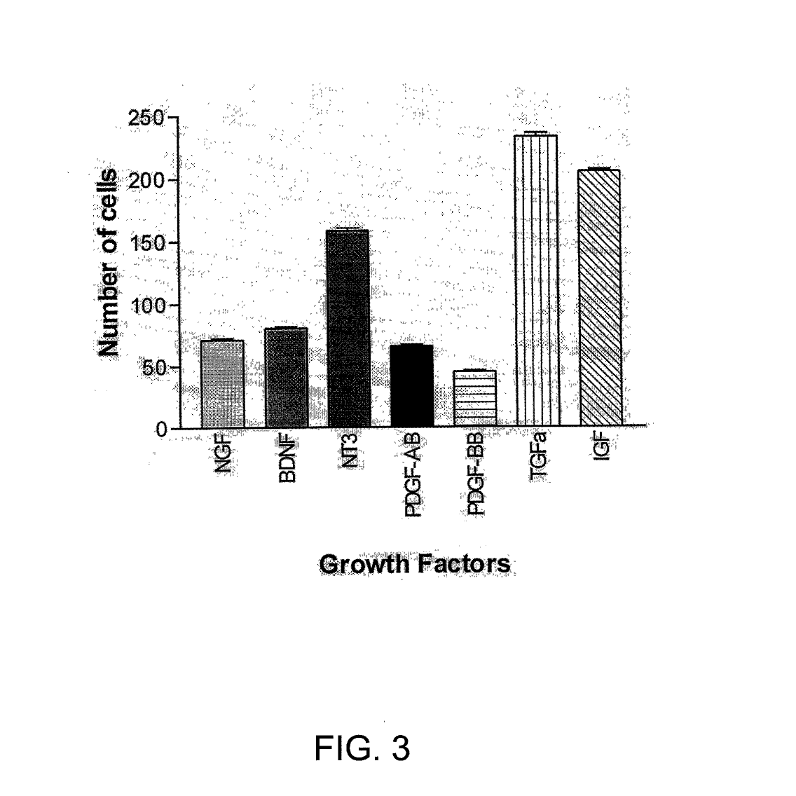

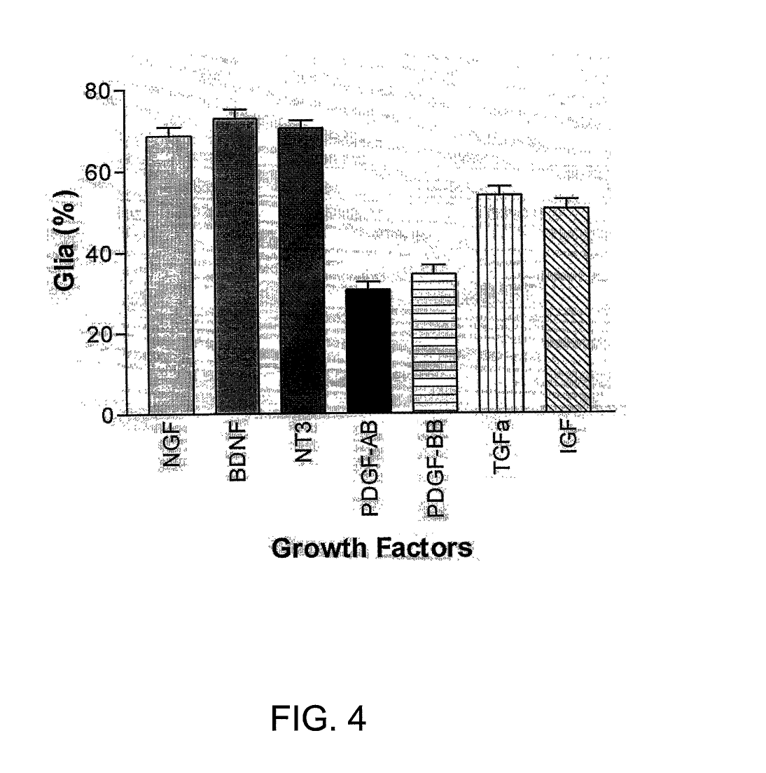

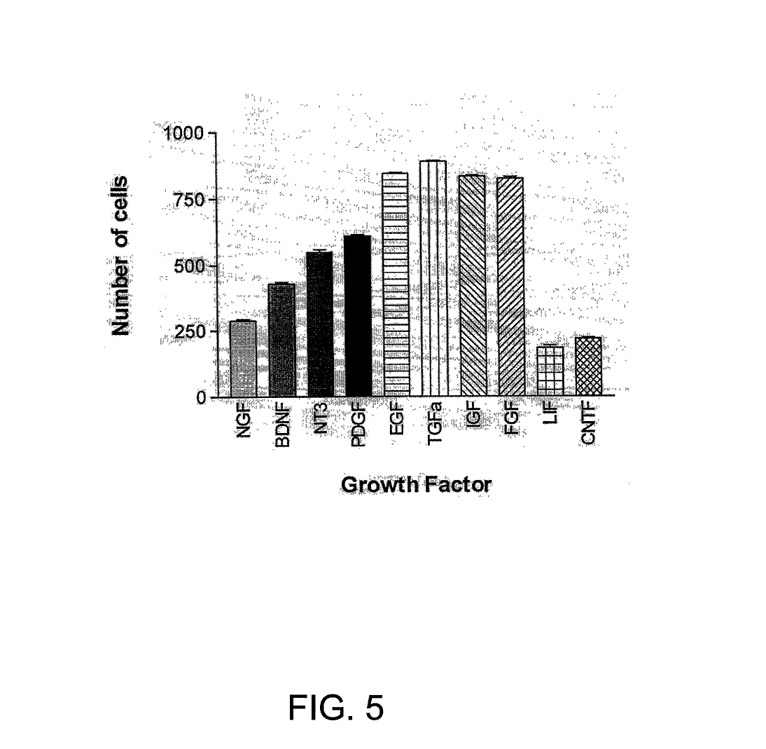

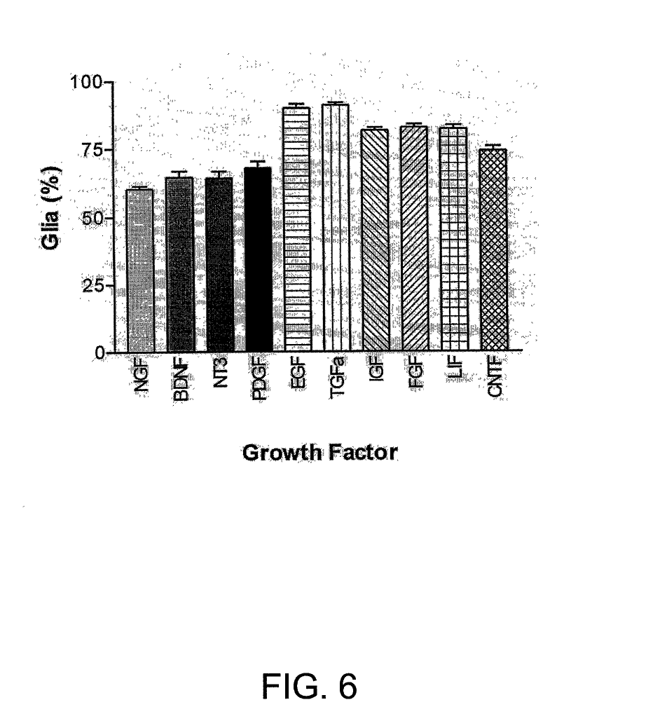

The highest numbers of cells and the best purification of ensheathing cells was obtained using DMEM supplemented with NT3 at 50 ng/ml (FIGS. 3 and 4) or Neurobasal Medium supplemented with TGFa (TGF α) (1 ng/ml) or EGF (10 ng/ml) (FIGS. 5 and 6) or combinations of EGF (10-100 ng/ml) and FGF2 (10-100 ng/ml).

Fetal calf serum (FCS) also appears to increase cell density, however, FCS also increases cell density of other non-ensheathing cells that may be present in the culture.

5. Expansion of Ensheathing Cells In Vitro

Once purified, ensheathing cells can be induced to proliferate using a forskolin-containing medium, as described by Ramon-Cueto (Ramon-Cueto et al, 1998, supra). It has also been found from lamina propria slice cultures that ensheathing cells were able to proliferate when co-cultivated with the other cell types present in the lamina propria. To recreate this environment, conditioned media was used. Unwanted cell types, collected after purification (for example, unbound cells during immuno-panning or magnetic separation) were centrifuged and cultivated in serum-containing medium on plastic dishes. Every two days, during the medium change, the supernatant was collected and used for feeding the cultures of purified ensheathing cells or frozen for future experiments. This method resulted in a significant increase of cell number and provides a means to propagate a purified ensheathing cell culture.

Additionally, there are a significant number of candidate growth factors which could affect ensheathing cell proliferation and survival as shown in FIGS. 3 to 6, which may be present in the conditioned media. Currently the ensheathing cells are known to express receptors for a variety of growth factors from the following families: EGF family, FGF family, neurotrophins, glial cell line-derived growth factor family (GDNF), PDGF family, cytokines, dopamine, and stem cell factor (SCF) as reviewed by Mackay-Sim and Chuah (Mackay-Sim and Chuah, 2000, Progress in Neurobiology 62 527), herein incorporated by reference.

Extracellular matrix molecules may also affect ensheathing cell proliferation and survival. The large differences in cell numbers between FIGS. 3 and 5 may be due in part to the difference in the substrates used to grow the cells (plastic versus fibronectin). Similarly the relative purities of the cultures (FIGS. 4 and 6) may in part be due to the same cause. Ensheathing cells secrete extracellular molecules such as laminin and heparan sulphate proteoglycans.

6. Grafting of Ensheathing Cells

The technique will differ according to the type of injury. Peripheral nerve-type injury and spinal cord-type injury can be distinguished. In spinal cord-type injury a cut or gap is usually absent and therefore transplant cells have to be inserted into the damaged area using micro-needles.

In peripheral nerve-type injury, there is usually a gap between the two stumps of the nerve. Therefore, a bridge (for example, a biodegradable polyglycolic acid tube) filled with the purified ensheathing cells is required. Since peripheral nerves also contain fibroblasts and endothelial cells which are present in the lamina propria, it is possible to use bridges filled with small pieces of purified lamina propria.

The therapeutic potential of olfactory ensheathing cells was tested on 10 rats in which a 17 mm section of the sciatic nerve was removed. The two stumps were bridged by a 20 mm silicon tube. In the experimental group (5 animals), the tube was filled with purified ensheathing cells resuspended in culture medium while in the control group (5 rats) the tube was filled only with culture medium. Two months later, the animals were sacrificed and the sciatic nerve observed. In 3 experimental animals out of 5, nerve fibers were found in the tube while no control animal showed any nerve regrowth.

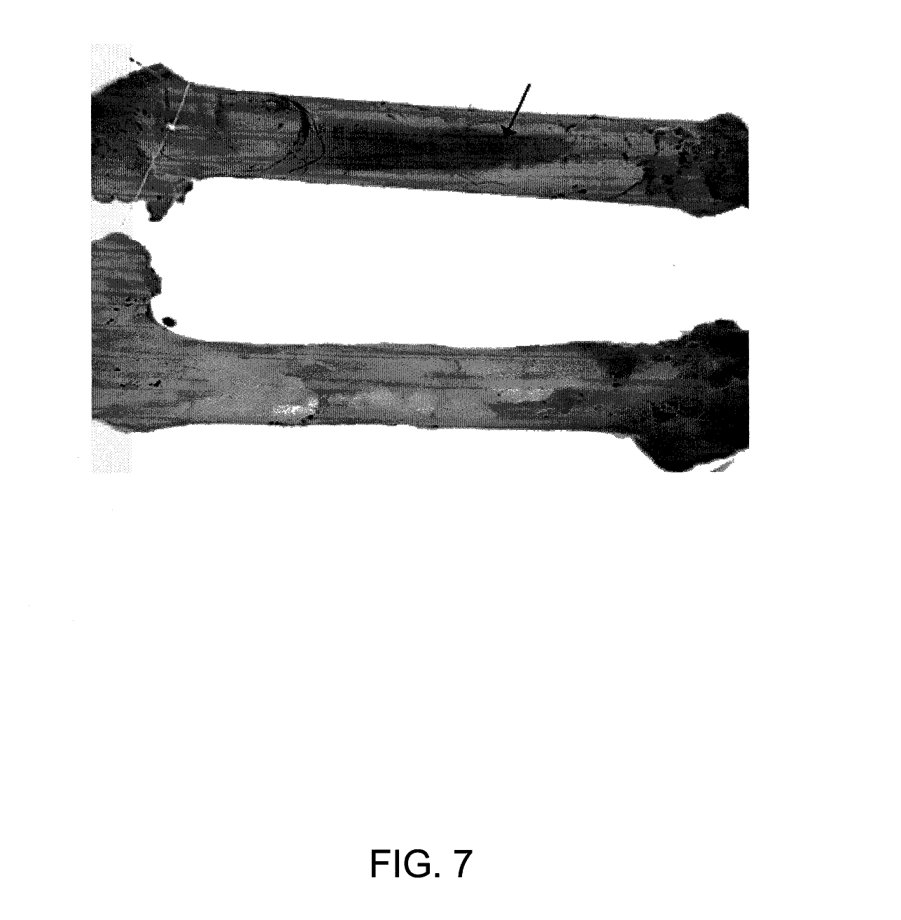

FIG. 7 shows nerve regrowth after ensheathing cell grafting. A 17 mm sciatic nerve gap was created and the two stumps were connected using a silicon tube filled with either culture medium (control group, bottom image) or purified ensheathing cells resuspended in culture medium (experimental group, top image).

7. Olfactory Lamina Propria Transplants Promote Behavioural Recovery after Spinal Transection in Rat

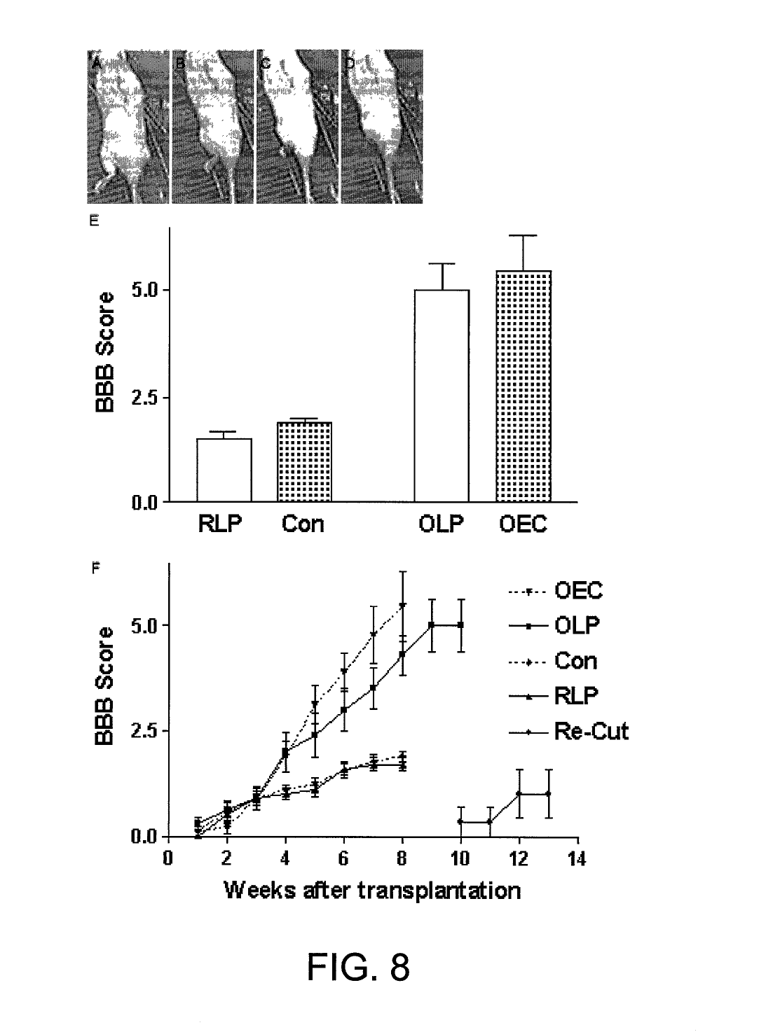

Lamina propria transplantation can promote behavioural recovery after complete spinal cord transection in the rat. Intact pieces of the lamina propria were transplanted into the transected spinal cord of rats to provide a source of olfactory ensheathing cells as well as acting as a bridge or physical support across the cut cord surfaces (FIG. 8). Adult female rats were anaesthetised with ketamine/rompun mixture (90/10 mg/kg, (IP) intraperitoneally) and the spinal cord completely transected at T10. Intact pieces of olfactory lamina propria (n=10) or respiratory lamina propria (n=10) were transplanted into the transected spinal cords respectively. Following surgery (up to 10 weeks), functional assessment of locomotor activity (BBB score) was performed blind as to treatment. Significant functional recovery in hind limb usage occurred in olfactory lamina propria-transplanted animals compared with controls, transplanted with respiratory lamina propria or collagen matrix respectively (FIG. 8). Olfactory lamina propria-treated rats developed the ability to sweep with the hind limb, in a motion that involved all three joints. By 8-10 weeks post-surgery 6 out of 10 animals grafted with olfactory lamina propria achieved a BBB score of 6-8 in one or both legs, with ankle, knee and hip movement and dorsiflexion of the foot (FIG. 8A-8D). None of the animals showed coordinated fore and hind limb movements or the ability to bear weight on the hind limbs. The maximal hind limb movement of controls after 10 weeks was limited to ankle or slight knee movement, with the foot plantar-flexed and dragged behind (BBB score, 0-2; scores in the control animals with respiratory lamina propria or collagen matrix were similar so results from both these groups were pooled). For olfactory lamina propria treated animals, improvements could occur in one or both hind limbs, with either side showing movements. The mean BBB score for the best leg for all animals (FIG. 8) was significantly higher in the olfactory transplant rats (5.0±1.9, range 2-8) compared to control animals (1.5±0.5, range 0-2; t=5.5, p<0.0001). When asymmetrical recovery occurred it was not obviously associated with asymmetrical reflex modulation or histological repair (see below), but was generally linked to an asymmetrical posture; most animals lay on one side with the recovered leg uppermost. The hind limb movement of the olfactory transplant rats began to significantly differ from the controls after 3 weeks, with continued divergence of the mean BBB score until 10 weeks (FIG. 8). Three animals with BBB scores of 4-6 were recut at 10 weeks to assess the effect on their functional recovery. One day after the retransection neither leg showed any movement (FIG. 8). Over the subsequent 2 weeks the BBB scores increased to 1-2 then remained stable at this level for a third week. This latter result indicates that the behavioural recovery of limb use depended upon regrowth of axons through the transection/graft site. Taken together, these experiments indicate that olfactory lamina propria transplants are very effective in promoting functional recovery after complete spinal cord transection.

8. Olfactory Lamina Propria Ensheathing Cell Transplants Promote Behavioural Recovery after Spinal Transection in Rat

The experiments above in part 7 were repeated using transplants of olfactory ensheathing cells derived from the lamina propria of olfactory mucosa. Use of ensheathing cells from the olfactory mucosa in transplantation is new and has the advantages as mentioned herein. Other studies have involved ensheathing cell transplants from the olfactory bulb, in contrast with the present invention whereby the ensheathing cells are isolated from the olfactory lamina propria. Studies using ensheathing cells from the olfactory bulb have shown some functional recovery after complete transection of the spinal cord (Ramon Cueto et al, 2000, Neuron 25 425). In addition it has been shown that human olfactory ensheathing cells can remyelinate axons in demyelinated rat spinal cord (Kato et al, 2000, Glia 30 209; Barnett et al, 2000, Brain 123 1581). As above, all rats which received olfactory ensheathing cell transplants recovered some hindlimb movement by 10 weeks, as measured by the BBB score (FIG. 8E and F). Control rats receiving no cells and only a collagen matrix did not recover hindlimb use (FIG. 8E and F). When compared to the experiments described above these results indicate that cell dissociation and purification is not a necessary prerequisite for behavioural recovery. Conversely, the results indicate that dissociated olfactory ensheathing cells from the olfactory lamina propria can promote behavioural recovery after spinal cord injury just as cells from the olfactory bulb are reported to. A two-way analysis of variance comparing the data from lamina propria transplants and olfactory ensheathing cell transplants (FIG. 8F) indicated no significant difference between transplant type (F1,34=0.638, p=0.42) whereas the effect of the transplant tissue (olfactory versus non-olfactory) was significant (F1,34=45.76, p<0.0001).

9. Olfactory Lamina Propria Transplants Promote Recovery of Inhibition of Spinal Reflex after Spinal Transection in Rat

Physiological Assessment of Reflexes

Reflex excitability was tested using a modification of the method reported by Skinner et al, 1996, Brain Research 729 127. The H-reflex responses to repetitive stimulation at 10 Hz is normally abolished by the second and subsequent stimuli, probably through presynaptic inhibitory mechanisms. However, in transected animals, this normal inhibition is absent, and the H-reflex amplitude remains close to 100% of its control value. The H-reflex excitability was assessed in 6 transected rats 10 weeks after olfactory lamina propria transplants, 6 transected control animals transplanted with respiratory lamina propria 9-10 weeks previously (n=4), or with collagen matrix 2-4 weeks before (n=2) and 5 normal control rats. Animals were anaesthetised with Ketamine and rompun and body temperature maintained as described above. Electromyographic activity (EMG) in the fourth dorsal interosseus muscle was recorded using a bipolar tungsten electrode, in response to stimulation of the lateral plantar nerve at the ankle. The signal was amplified using a differential amplifier and recorded using the Maclab system (AD Instruments Pty. Ltd., Castle Hill, NSW, Australia). Single square wave stimuli (0.5 ms, 5-15V) were used to elicit the M-wave (direct muscle response) and H-reflex and then trains of 5 stimuli at 10 Hz were delivered at 5× H-reflex threshold. The amplitude of the M-wave was monitored throughout to ensure it remained constant. H-reflex amplitude of the second response was measured from the average of 3 trials and expressed as a percentage of the first response, also averaged over 3 trials. The profiles of subsequent responses (3^{rd}-5^{th}) were used to assess stability of the reflex depression. H-reflex amplitudes in normal, control and olfactory lamina propria-transplanted animals were compared using ANOVA.

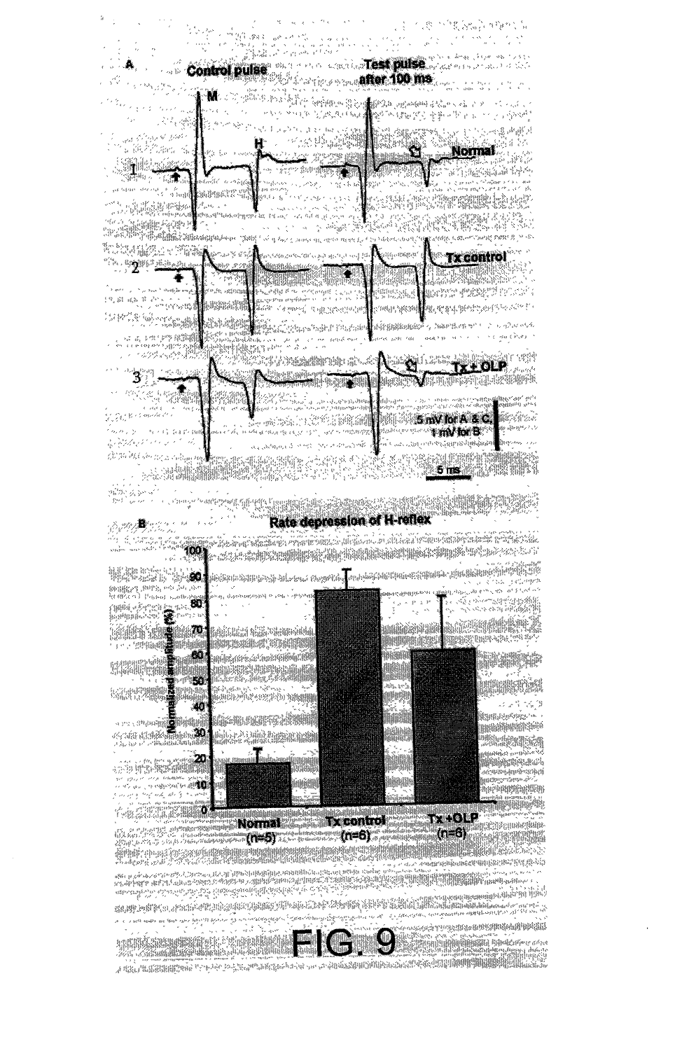

Examples of EMG activity in the fourth dorsal interosseous muscle following stimulation of the lateral plantar nerve stimulation are shown in FIG. 9. In each case the response consists of the M-wave, the EMG elicited by direct stimulation of motor axons, followed by the H-reflex, the EMG elicited indirectly by stimulation of the sensory axons. In normal animals, stimulation at 10 Hz resulted in a marked reduction in the H-reflex amplitude for the second and subsequent stimuli (17+6%, normalised to the first response, FIG. 9), as has been noted before Skinner et al, 1996, supra. This rate-sensitive depression is absent in transected animals and was not seen here in rats transplanted with respiratory lamina propria (83+8%). However, olfactory lamina propria-transplanted animals showed an intermediate level of reflex depression (59+20%). While there was considerable variability in individual animals, the mean value was significantly different from both normal (p<0.01) and transected control rats (p<0.05).

10. Olfactory Lamina Propria Transplants Promote Regrowth of Spinal Axons Across a Graft Site after Spinal Transection in Rat

Retrograde Labelling of Axons Crossing a Transplantation Site

After a survival period of 8-10 week, rats were anaesthetised as described above and the spinal cord was exposed below the lesion at the T11 level. Fluororuby (10% of dextran tetramethylrhodamine; 10000 M_{w}; Molecular Probes Inc.) was injected into the cord at the T11 level, using a Hamilton syringe. Three syringe placements were made, at the midline and 1 mm lateral on each side, to penetrate the dorsal columns and corticospinal tract, and the ventrolateral and dorsolateral funiculi. For each placement, 3 pressure injections of Fluororuby (0.05 μl each at 1.5 mm, 1 mm and 0.5 mm deep) were made over a period of 3 minutes. Following a post-injection survival period of 2 to 4 days the rats were anaesthetised as described above and intracardially perfused with heparinised physiological saline followed by 4% paraformaldehyde in 0.1 M phosphate buffer (pH 7.4). The spinal cord extending from 5 mm rostral to 5 mm distal to the transection site, together with the brainstem, was removed, post-fixed for 2 hours in the same fixative, cryoprotected in 30% sucrose overnight and prepared for cryo-sectioning. The spinal cord was sectioned longitudinally and the brainstem coronally at 50-100 μm. Fluorescent tissues were observed with confocal laser microscopy.

Immunohistochemistry

Following incubation with 5% bovine serum albumin in phosphate buffered saline (PBS) for 30 min, monoclonal antibody to neurofilament 200 kDa (NF, Sigma Co., St. Louis, Mo., diluted 1:400 in 0.1 M PBS, pH 7.4) was used as a primary antiserum to detect nerve fibres at the lesion site. After 4 hours of incubation at room temperature, sections were washed and incubated in secondary antibody (biotinylated horse anti-mouse, Vector Laboratories Inc., diluted 1:200 with PBS plus 0.5% Triton X-100, PBST) for 1 hour followed by the Vector ABC procedure for peroxidase staining and visualisation with 3,3′-diaminobenzidine (DAB). The specificity of the immunostaining for neurofilament was verified by omission of primary antibody.

Selected sections were processed for serotonin immunostaining of fibres in the grafting site and the adjacent cord. After the blocking step in 5% normal goat serum, the sections were incubated in primary antibody at 4° C. overnight (rabbit, DiaSorin Inc; diluted 1:1000 in PBS). The following day, sections were washed with PBS and incubated with the secondary antibody (biotinylated goat-anti-rabbit IgG, Sigma Co.; diluted 1:200 in PBST) for 1 hour. The sections were then reacted with ABC reagent with DAB as chromogen to visualize the 5-HT positive axons. Rat brainstem raphe neurons were used in staining as positive controls for the specificity of the anti-serotonin antibody, and first antibody was omitted for negative controls.

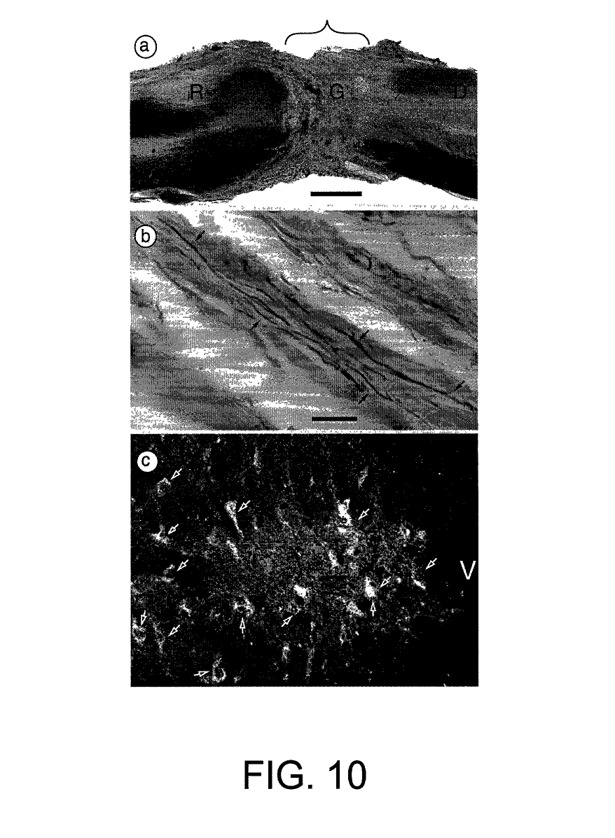

The olfactory lamina propria grafts integrated very well into the damaged spinal cord (FIG. 10a). Grafts pre-labelled with Cell Tracker green showed graft cells penetrating into the rostral and caudal spinal cord stumps for up to 3.5 mm and many were still present within the graft 10 weeks after transplantation (not shown). Axons penetrating the graft were identified using anti-neurofilament immunoreactivity and many were seen clearly within the graft (FIG. 10b) and entering the rostral and caudal spinal cord. Injections of Fluororuby were made into the spinal cord caudal to the graft site. This was retrogradely transported through the graft and into cell bodies located into the nucleus raphe magnus in the brain stem (FIG. 10c).

In control spinal cords with grafts of either respiratory lamina propria or collagen matrix, there were no neurofilament-positive axons in the graft and no Fluororuby labelled cells in the nucleus raphe magnus. Fluororuby labelled axons extended up to the distal edge of the graft but were never observed to penetrate the graft site. The two animals with olfactory lamina propria transplants which showed no behavioural recovery (BBB score 2) also showed no histological evidence of axonal regeneration.

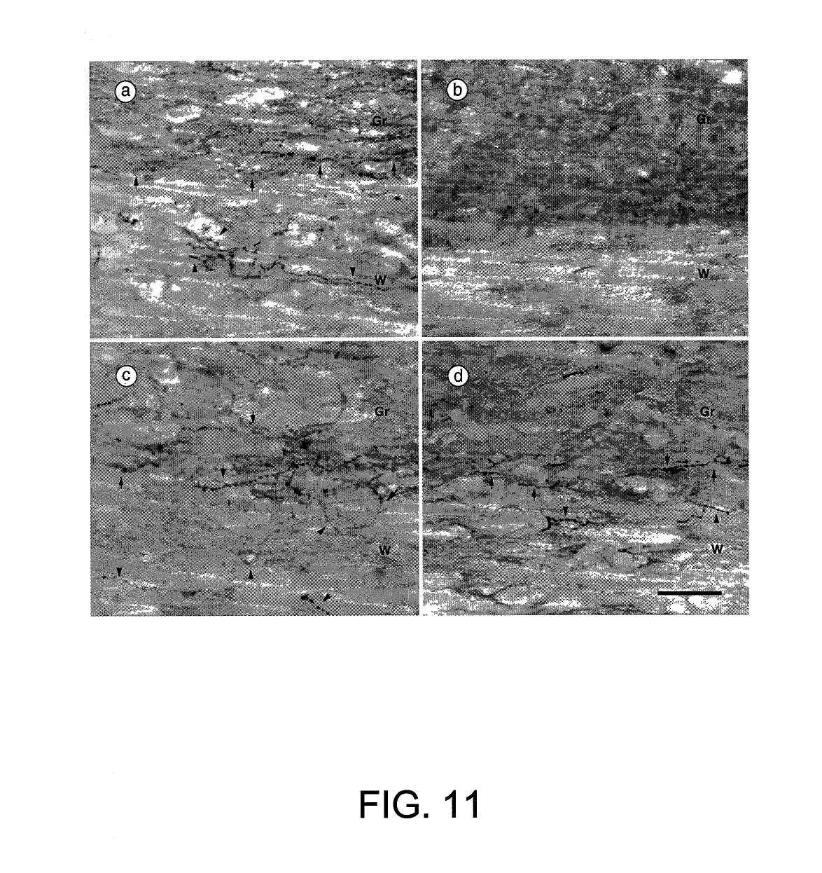

Serotonergic fibres in the spinal cord arise from the brainstem raphe nuclei (Tork, 1985, in G. Paxinos (Ed), The rat nervous system; hindbrain and spinal cord, pp 43-78). As expected, numerous serotonin-immunoreactive fibres were observed in the grey and white matter of the spinal cord rostral to both olfactory lamina propria grafts and respiratory lamina propria grafts (FIGS. 11a and 11c). However, only after olfactory lamina propria transplants were serotonergic fibres seen within the transplant site and within the spinal cord caudal to the graft (FIG. 11d); these fibres were not present in control animals (FIG. 11b). In the olfactory lamina propria transplanted animals, serotonergic axons were observed at least 6 mm caudal to the graft. They were mostly present in the grey matter of the ventral cord, and along the border zone between the grey and white matter, but a few were also present within the white matter.

11. Olfactory Lamina Propria Autologous Transplant after Spinal Transection in Monkey

The spinal cords of two monkeys were hemisectioned at T10 and autologous transplants of olfactory mucosa were performed. Three months after the surgery, these two animals could flex all joints except the toes on the affected leg. One can voluntarily use its leg. A control animal (hemisectioned without transplantation) showed no such recovery before it had to be sacrificed because of an unrelated infection. A second control animal recovered the use of the affected limb without olfactory lamina propria transplantation. Recovery from similar hemisectioning of the spinal cord would not be seen in humans and we have no explanation for our results without further experimentation.

In summary, it is appreciated that olfactory ensheathing cell and lamina propria transplants of the present invention show great potential for therapeutic intervention after spinal injury and nerve regeneration of the facial and trigeminal nerves after surgical removal of carcinomas of the head and neck. Therapeutic intervention which could lead to the recovery of function after severe spinal injury or surgery would clearly have many very significant medical and social consequences. Even limited use of limbs or limited control over bodily functions would have major consequences for individuals in their daily lives.

It will be understood that the invention described in detail herein is susceptible to modification and variation, such that embodiments other than those described herein are contemplated which nevertheless falls within the broad spirit and scope of the invention.

1. A method of isolating ensheathing cells comprising the steps of:
(i) isolating olfactory mucosa;
(ii) isolating lamina propria from the isolated olfactory mucosa; and
(iii) isolating ensheathing cells from the isolated lamina propria.

2. The method of claim 1 whereby the olfactory mucosa is isolated from dorso-medial area of a nasal septum, superior turbinate or middle turbinate proximal to the cribriform plate.

3. The method of claim 1 whereby the olfactory mucosa is isolated from an adult.

4. The method of claim 1 whereby the olfactory mucosa is isolated from a mammal.

5. The method of claim 4 whereby the mammal is a human.

6. The method of claim 1 wherein step (i) includes the steps of:
(a) enzymatic digestion of the isolated olfactory mucosa; and
(b) mechanical separation of the lamina propria from the enzymatically digested isolated olfactory mucosa of step (a).

7. The method of claim 6 wherein step (a) includes use of dispase II.

8. The method of claim 7 wherein the dispase II concentration is in a range of 2.0 to 3.0 units/ml.

9. The method of claim 6 whereby the mechanical separation of step (b) is by microscopic dissection.

10. The method of claim 1 including the steps of:
(i) enzymatically digesting the isolated lamina propria of step (ii); and
(ii) isolating ensheathing cells from the enzymatically digested isolated lamina propria of step (i).

11. The method of claim 10 whereby step (i) includes using collagenase L and dispase II.

12. The method of claim 10 whereby step (i) includes using collagenase L.

13. The method of claim 1 wherein step (ii) includes the steps of:
(A) culturing the isolated lamina propria of step (ii); and
(B) allowing ensheathing cells to migrate away from the cultured lamina propria.

14. The method of claim 13 whereby the isolated lamina propria is a 200-400 mm thick slice.

15. The method of claim 1 wherein the step of isolating the ensheathing cells includes isolating ensheathing cells bound by an antibody.

16. The method of claim 15 including the steps of immuno-panning, immunoprecipitation or a combination thereof.

17. The method of claim 16 whereby immunoprecipitation includes the step of using magnetic beads whose surface is coated with a secondary antibody that binds to the antibody that binds the ensheathing cells.

18. The method of claim 15 wherein the antibody that binds ensheathing cells is a monoclonal antibody that binds p75.

19. The method of claim 15 further including the step of culturing the antibody bound ensheathing cells in a culture medium supplemented with at least one of the following: epidermal growth factor, basic fibroblast growth factor, brain-derived neurotrophic factor, neurotrophic growth factor, neurotrophin 3, platelet-derived growth factor A or platelet-derived growth factor B, transforming growth factor a, leukemia inhibitory factor, ciliary neurotrophic factor or insulin-like growth factor-I.

20. A method of expanding a culture of ensheathing cells including the steps of co-cultivation of ensheathing cells with cells from the lamina propria.

21. The method of claim 20 whereby the cells from the lamina propria comprise cells which do not bind to antibodies which bind to ensheathing cells.

22. A method of expanding a culture of ensheathing cells including the steps of culturing ensheathing cells in conditioned medium from lamina propria cell culture.

23. The method of claim 22 wherein the conditioned medium is medium collected from cell cultures of lamina propria cells which do not bind to antibodies which bind to ensheathing cells.

24. The method of any one of the preceding claims including the step of transplanting the isolated ensheathing cells to a recipient.

25. A method of isolating lamina propria including the steps of:
(i) isolating olfactory mucosa from a human; and
(ii) isolating lamina propria from the isolated olfactory mucosa.

26. A method of transplantation including the steps of:
(i) isolating olfactory lamina propria from olfactory mucosa of a donor; and
(ii) transplanting the isolated olfactory lamina propria of step (I) to a recipient.

27. The method of claim 26 wherein the lamina propria is intact.

28. The method of claim 26 wherein the lamina propria is dissociated.

29. The method of claim 26 whereby the transplantation is heterologous or autologous.

30. The method of claim 26 whereby the transplantation is autologous.

31. The method of claim 26 whereby the donor or recipient is an animal.

32. The method of claim 31 whereby the animal is a mammal.

33. The method of claim 32 whereby the mammal is a human.

34. The method of claim 26 whereby the transplantation is to any organ or tissue of the recipient capable of neural growth.

35. The method of claim 34 whereby the organ or tissue has nerve damage.

36. The method of claim 34 or claim 35 whereby the organ or tissue is selected from the group consisting of brain, spine and peripheral nerves.
